(19) United States
(12) Patent Application Publication
(10) Pub. No.: US 2025/0017927 A1
(43) Pub. Date: Jan. 16, 2025

(54) PHARMACEUTICAL COMPOSITION AND USE THEREOF

(71) Applicant: SHANGHAI ALLIST PHARMACEUTICALS CO., LTD., Shanghai (CN)

(72) Inventors: Huibing LUO, Shanghai (CN); Qing LI, Shanghai (CN)

(21) Appl. No.: 18/712,357

(22) PCT Filed: Nov. 21, 2022
(86) PCT No.: PCT/CN2022/133171

§ 371 Date: May 22, 2024

(30) Foreign Application Priority Data
Nov. 24, 2021 (CN) 202111404293.6

(51) Int. Cl. A61K 31/506; A61K 9/00; A61P 35/00

(52) U.S. Cl. CPC A61K 31/506; A61K 9/0053; A61P 35/00

(57) ABSTRACT

The present disclosure provides a pharmaceutical composition containing a therapeutically effective amount of furmonertinib or a pharmaceutically acceptable salt thereof and optionally a pharmaceutically acceptable carrier, and use of furmonertinib or a pharmaceutically acceptable salt thereof, or said pharmaceutical composition in manufacture of a medicament for treating and/or preventing a disease mediated by HER2 exon 20 insertion mutation and/or EGFR rare mutation. The pharmaceutical composition of the present disclosure shows an excellent therapeutic effect on disease mediated by HER2 exon 20 insertion mutation and/or EGFR rare mutation (for example, non-small cell lung cancer (NSCLC)) with little side effects and excellent safety.

### TECHNICAL FIELD

The present disclosure relates to a pharmaceutical composition comprising a therapeutically effective amount of furmonertinib or a pharmaceutically acceptable salt thereof and optionally a pharmaceutically acceptable carrier. The present disclosure also relates to use of furmonertinib or a pharmaceutically acceptable salt thereof, and the pharmaceutical composition in manufacture of a medicament for treating and/or preventing a disease mediated by human epidermal growth factor receptor-2 (HER2) exon 20 insertion (HER2 Exon 20 insertion) mutation (hereinafter sometimes referred to as HER2 Exon 20 insertion mutation) and/or epidermal growth factor receptor (EGFR) rare mutation (hereinafter sometimes referred to as EGFR rare mutation). The present disclosure also provides a method of treating and/or preventing a disease mediated by HER2 exon 20 insertion mutation and/or EGFR rare mutation, comprising administering to a patient a therapeutically effective amount of furmonertinib or a pharmaceutically acceptable salt thereof.

### BACKGROUND TECHNOLOGY

Worldwide, lung cancer has always been a malignant tumor with the highest morbidity and mortality and serious harm to human health and life, and 1.76 million people died of lung cancer in 2018 all over the world. Non-small cell lung cancer (NSCLC) comprises approximately 80-85% of all lung cancers. Epidermal Growth Factor Receptor (EGFR) is a multifunctional glycoprotein widely distributed on the cell membrane of various tissues of the human body, and it is a member of the ERBB receptor family, which includes EGFR (HER1 or ERBB1), HER2 (ERBB2), HER3 (ERBB3) and HER4 (ERBB4) four members. The EGFR mutation is the most widely studied target in NSCLC.

Among EGFR mutations, common mutations include sensitive mutations (such as exon 19 deletion and exon 21 point mutation (L858R), comprising 85%-90% of all EGFR mutations), drug resistant mutations (such as exon 20 T790M mutation, exon 20 C797S mutation), etc.; rare mutations include EGFR G719S mutation, EGFR S768I mutation, EGFR G724S mutation, EGFR L861Q mutation, EGFR G719S/T263P mutation, etc.; in addition, EGFR mutation also includes EGFR exon 20 insertion mutation (approximately comprising 1-10% of all types of EGFR mutations).

Over the years, a large number of targeted drugs have been developed for EGFR mutation in NSCLC, such as the first generation of reversible tyrosinase inhibitor (TKI) Gefitinib and Erlotinib for EGFR sensitive mutation, the second generation of irreversible covalent binding inhibitor Afatinib, and the third generation of inhibitor Osimertinib for drug resistant mutation EGFR T790M, which have very good clinical effects.

HER2, another member of the ERBB family, is amplified and mutated in a variety of cancers. Among them, HER2 mutations comprise about 4% in NSCLC, and about 90% of HER2 mutations are exon 20 insertion mutations. Exon 20 of HER2 contains two major regions, the c-helix (residues 770 to 774) and the loop following the c-helix (residues 775 to 783 in HER2). The most common HER2 exon 20 insertion mutation is ERBB2 A775_G776insYVMA mutation, and less common ones are ERBB2 V777_G778insGC mutation, ERBB2 P780_Y781insGSP mutation, etc. Exon 20 insertion mutations result in increased HER2 kinase activity and enhanced signaling through downstream pathways, resulting in increased survival, invasiveness, and tumorigenicity. Tumors with the ERBB2 A775_G776insYVMA mutation are substantially resistant to known EGFR inhibitors. Currently, no small molecule targeted drug against HER2 exon 20 insertion mutation is approved globally.

In recent years, compounds that inhibit EGFR mutations and/or HER2 mutations (especially HER2 exon 20 insertion mutations) have been extensively studied. However, how to further improve the activity and reduce the toxic and side effects is still an existing problem.

N-{2-{[2-(dimethylamino)ethyl](methyl)amino}-6-(2,2,2-trifluoroethoxy)-5-{[4-(1-methyl-1H-indol-3-yl)pyrimidin-2-yl]amino}pyridin-3-yl}acrylamide (also referred to as “furmonertinib”) represented by the following formula (I) is described in patent CN105315259B, and the mesilate of the compound represented by the following formula (I) (also referred to as “furmonertinib mesilate”) is described in patent CN107163026B, and furmonertinib mesilate has been commercialized as a third-generation EGFR-TKI inhibitor, and is mainly used for treating a disease mediated by EGFR-sensitive mutation and T790M drug-resistant mutation. The phase I dose escalation study of furmonertinib mesilate demonstrates that when furmonertinib mesilate is orally taken once per day at a dosage level of 20 mg-240 mg, the tolerance and the safety are good, adverse events of subjects are mild or moderate, dose-limiting toxicity does not occur, and dose-related toxic reaction does not occur; and the phase IIb clinical trial has demonstrated that the oral administration of 80 mg daily dose of furmonertinib mesilate shows a relatively good anti-tumor effect on patients with the EGFR T790M positive advanced non-small cell lung cancer, who has progressive disease after receiving prior systematic anti-tumor therapy, and can alleviate or stabilize the disease progression.

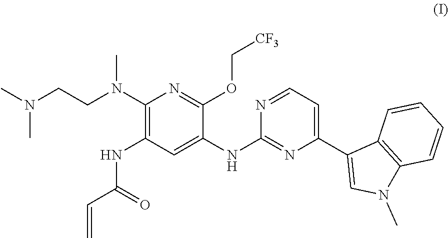 [SMILES: C=CC(=O)Nc1cc(Nc2nccc(-c3cn(C)c4ccccc34)n2)c(OCC(F)(F)F)nc1N(C)CCN(C)C]

### SUMMARY

The present disclosure provides, in some embodiments, use of furmonertinib or a pharmaceutically acceptable salt thereof.

In some embodiments, furmonertinib or a pharmaceutically acceptable salt thereof as an active compound can effectively inhibit HER2 exon 20 insertion mutation and/or EGFR rare mutation, and thus, furmonertinib or a pharmaceutically acceptable salt thereof can be used for treating and/or preventing a disease mediated by HER2 exon 20 insertion mutation and/or EGFR rare mutation.

Thus, in some embodiments, the present disclosure provides use of furmonertinib or a pharmaceutically acceptable salt thereof in manufacture of a medicament for treating and/or preventing a disease mediated by HER2 exon 20 insertion mutation and/or EGFR rare mutation.

In some embodiments, the present disclosure provides use of furmonertinib or a pharmaceutically acceptable salt thereof in combination of at least one second therapeutic agent in manufacture of a medicament for treating and/or preventing a disease mediated by HER2 exon 20 insertion mutation and/or EGFR rare mutation.

In some embodiments, furmonertinib or a pharmaceutically acceptable salt thereof is useful as an active compound at a certain dose, a disease mediated by HER2 exon 20 insertion mutation and/or EGFR rare mutation, particularly non-small cell lung cancer, can be treated and/or prevented, and the treatment and/or prevention of the disease are/is accompanied by little side effects and is excellent in safety.

More specifically, the present disclosure provides a pharmaceutical composition comprising a therapeutically effective amount of furmonertinib, or a pharmaceutically acceptable salt thereof, and optionally a pharmaceutically acceptable carrier.

The present disclosure also provides use of the above-mentioned pharmaceutical composition of the present disclosure in manufacture of a medicament for treating and/or preventing a disease mediated by the HER2 exon 20 insertion mutation and/or EGFR rare mutation.

The composition of the present disclosure is present in a formulation form of a tablet or a capsule, and in each unit formulation, the content of furmonertinib or a pharmaceutically acceptable salt thereof is 10 mg-400 mg.

When the pharmaceutical composition of the present disclosure is used for treating and/or preventing a disease mediated by HER2 exon 20 insertion mutation and/or EGFR rare mutation, the daily dose of furmonertinib or a pharmaceutically acceptable salt thereof may be 80 mg-400 mg. At this time, by adjusting the amount of the above-mentioned tablets or capsules, the daily dose of furmonertinib or a pharmaceutically acceptable salt thereof can be easily adjusted.

The present disclosure also provides a method of treating and/or preventing a disease mediated by HER2 exon 20 insertion mutation and/or EGFR rare mutation, comprising administering to a patient in need of a therapeutically effective amount of furmonertinib or a pharmaceutically acceptable salt thereof.

In the above treatment method of the present disclosure, it is desirable that the daily dose of furmonertinib or a pharmaceutically acceptable salt thereof is 80 mg-400 mg.

The present disclosure also provides a method of treating and/or preventing a disease comprising administering to a patient with positive HER2 exon 20 insertion mutation and/or EGFR rare mutation a therapeutically effective amount of furmonertinib or a pharmaceutically acceptable salt thereof.

The present disclosure also provides a method of treating locally advanced or metastatic non-small cell lung cancer (NSCLC) comprising administering to a patient in need thereof a therapeutically effective amount of furmonertinib or a pharmaceutically acceptable salt thereof.

The present disclosure also provides a method of treating locally advanced or metastatic non-small cell lung cancer (NSCLC) comprising administering to a patient with confirmed positive HER2 exon 20 insertion mutation and/or EGFR rare mutation a therapeutically effective amount of furmonertinib or a pharmaceutically acceptable salt thereof.

The present disclosure also provides a method of treating locally advanced or metastatic non-small cell lung cancer (NSCLC) comprising administering to a patient harboring HER2 exon 20 insertion mutation and/or EGFR rare mutation a therapeutically effective amount of furmonertinib or a pharmaceutically acceptable salt thereof.

The present disclosure also provides a method of treating locally advanced or metastatic non-small cell lung cancer (NSCLC) comprising administering to a patient with confirmed positive HER2 exon 20 insertion mutation and/or EGFR rare mutation who has received no prior systematic anti-tumor therapy a therapeutically effective amount of furmonertinib or a pharmaceutically acceptable salt thereof.

The present disclosure also provides a method of treating locally advanced or metastatic non-small cell lung cancer (NSCLC) comprising administering to a patient with confirmed positive HER2 exon 20 insertion mutation and/or EGFR rare mutation who has progressive disease after receiving prior systematic anti-tumor therapy a therapeutically effective amount of furmonertinib or a pharmaceutically acceptable salt thereof.

In some embodiments, furmonertinib or a pharmaceutically acceptable salt thereof, or a pharmaceutical composition comprising furmonertinib or a pharmaceutically acceptable salt thereof and optionally a pharmaceutically acceptable carrier, exhibits excellent inhibitory activity against HER2 exon 20 insertion mutation and/or EGFR rare mutation, and therefore, it can show excellent clinical effect.

In addition, when furmonertinib or a pharmaceutically acceptable salt thereof, or a pharmaceutical composition comprising furmonertinib or a pharmaceutically acceptable salt thereof and optionally a pharmaceutically acceptable carrier of the present disclosure is used for treating and/or preventing a disease mediated by HER2 exon 20 insertion mutation and/or EGFR rare mutation, the side effect is small and the safety is excellent.

The pharmaceutical composition of the present disclosure can be prepared into a formulation having an appropriate size and an appropriate content of active components by containing furmonertinib or a pharmaceutically acceptable salt thereof in a specific amount.

### DETAILED DESCRIPTION

Embodiments of the present disclosure will be described in more detail below with reference to specific embodiments, but those skilled in the art will appreciate that the specific embodiments described below are merely illustrative of the present disclosure and should not be construed as limiting the scope of the present disclosure. On the contrary, the present disclosure is intended to cover all alternatives, modifications and equivalents, which may be included within the scope of the present disclosure as defined by the appended claims.

Unless otherwise specified, the embodiments of the present disclosure may be combined in any manner, and the conversions, modifications, and changes of the technical solutions obtained thereby are also included in the scope of the present disclosure.

Furmonertinib is a compound known in the prior art, described in particular in patent CN105315259B, with the chemical name: N-{2-{[2-(dimethylamino)ethyl](methyl)amino}-6-(2,2,2-trifluoroethoxy)-5-{[4-(1-methyl-1H-indol-3-yl)pyrimidin-2-yl]amino}pyridin-3-yl}acrylamide; the structural formula is the compound shown in the formula (I).

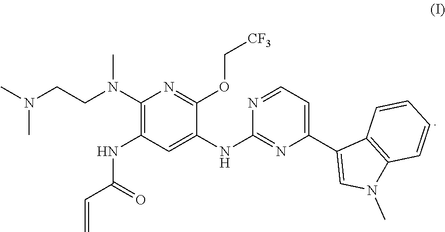 [SMILES: C=CC(=O)Nc1cc(Nc2nccc(-c3cn(C)c4ccccc34)n2)c(OCC(F)(F)F)nc1N(C)CCN(C)C]

In some embodiments, the active component for the treatment of the disease is actually furmonertinib or a pharmaceutically acceptable salt thereof. Therefore, in some embodiments, furmonertinib or a pharmaceutically acceptable salt thereof may be used alone or may be used by being contained in a composition, in which case the composition may optionally include a pharmaceutically acceptable carrier as desired.

In addition, in some embodiments, furmonertinib or a pharmaceutically acceptable salt thereof can also be used in combination with at least one second therapeutic agent.

The present disclosure provides a pharmaceutical composition comprising a therapeutically effective amount of furmonertinib or a pharmaceutically acceptable salt thereof and optionally a pharmaceutically acceptable carrier.

“Pharmaceutically acceptable carrier” means one or more compatible solid or liquid fillers or gelatinous materials which are suitable for human use and should be of sufficient purity and sufficiently low toxicity. The carrier is also known as “adjuvant”. “Compatibility” means that each component in the composition can be admixed with the compounds of the present disclosure and with each other without significantly reducing the drug effect of the compounds. Some examples of pharmaceutically acceptable carriers include cellulose and derivatives thereof (such as sodium carboxymethyl cellulose, ethyl cellulose, methyl cellulose, hydroxypropylmethyl cellulose and derivatives thereof, cellulose acetate and derivatives thereof, cellulose acetate, etc.), gelatin, talc, solid lubricants (such as stearic acid, magnesium/calcium stearate, hydrogenated vegetable oil, sodium stearyl fumarate), calcium sulfate, vegetable oils (such as soybean oil, sesame oil, peanut oil, olive oil, etc.), polyols (such as propylene glycol, glycerol, mannitol, sorbitol, etc.), emulsifiers, wetting agents (such as sodium dodecyl sulfate), coloring agents, flavoring agents, stabilizers, antioxidants, preservatives, etc, but not limited thereto.

The pharmaceutical compositions may be prepared by methods well known in the art, such as conventional mixing, dissolving, granulating, dragee-making, levigating, emulsifying, and lyophilizing processes.

The pharmaceutical compositions may be present in the formulation form of a tablet or a capsule, in the formulation, furmonertinib or a pharmaceutically acceptable salt thereof is mixed with at least one pharmaceutically acceptable carrier, in the present disclosure, the carrier is also known as “adjuvant”, said carrier may include but not limited to: (a) fillers or solubilizing agents, for example, microcrystalline cellulose, starch, lactose, sucrose, glucose, mannitol, colloidal silica, calcium hydrogen phosphate, calcium phosphate, calcium sulfate; (b) binders, for example, hydroxypropylmethylcellulose, hydroxypropylcellulose, methylcellulose, alginates, gelatin, polyvinylpyrrolidone, copovidone, sucrose and acacia, corn starch; (c) humectants, for example glycerin and the like; (d) disintegrants, for example, croscarmellose sodium, crospovidone, sodium carboxymethyl starch, colloidal silica, microcrystalline cellulose, potato starch or tapioca starch or corn starch, pregelatinized starch, alginic acid, certain complex silicates and sodium carbonate, ion exchange resins and the like; (e) absorption accelerators, for example, quaternary ammonium compounds, anionic or nonionic surfactants, cyclodextrins, and the like; (f) wetting agents such as cetyl alcohol and glycerol monostearate and the like; (g) adsorbents, for example, kaolin, colloidal silica, ion exchange resins, and the like; and (h) lubricants, for example, talc, calcium stearate, magnesium stearate, solid polyethylene glycols, sodium lauryl sulfate, sodium stearyl fumarate, hydrogenated vegetable oils, and the like, or mixtures thereof. The capsule and the tablet may also contain buffering agent. Tablets and capsules may be coated or microencapsulated with a coating or shell material such as an enteric coating or other materials known in the art.

The term “pharmaceutically acceptable salt” is a salt prepared from furmonertinib and a relatively non-toxic, pharmaceutically acceptable acid or base. Base addition salts may be obtained by contacting furmonertinib with a sufficient amount of a pharmaceutically acceptable base in pure solution or in a suitable inert solvent. Representative base addition salts include, for example, those salts formed with alkali metal, alkaline earth metal, quaternary ammonium cations such as sodium, lithium, potassium, calcium, magnesium, tetramethylquaternary ammonium, tetraethylquaternary ammonium, and the like; amine salts, including salts formed with ammonia (NH₃), primary, secondary or tertiary amines, such as methylamine salts, dimethylamine salts, trimethylamine salts, triethylamine salts, ethylamine salts, and the like. In addition, acid addition salts may be obtained by contacting furmonertinib with a sufficient amount of a pharmaceutically acceptable acid in pure solution or in a suitable inert solvent. The pharmaceutically acceptable acid salt comprises inorganic acid salts such as hydrochloride, sulfate, phosphate, and nitrate; and organic acid salts such as formate, acetate, propionate, methanesulfonate, benzylsulfonate, succinate, citrate, and tartrate. Reference can be specifically made to Berge et al., “Pharmaceutical Salts”, Journal of Pharmaceutical Science 66: 1-19 (1977), or “Handbook of Pharmaceutical Salts: Properties, Selection, and Use” (P. Heinrich Stahl and Camille G. Wermuth, ed., Wiley-VCH, 2002).

As used herein, “therapeutically effective amount” refers to a sufficient amount of drug or pharmacologically active agent that is non-toxic but yet achieves the desired effect. The effective amount will vary from person to person, depending on the age, weight and condition of the patient and also on the particular active substance, and an appropriate effective amount in individual cases may be determined by a person skilled in the art in the light of routine test.

As used herein, “active component”, “active substance”, or “active agent” refers to a chemical entity that is effective in treating the disorder, disease, or condition of interest.

As used herein, “patient”, “individual”, or “subject” includes humans, animals, vertebrates, mammals, rodents (e.g., guinea pigs, hamsters, rats, mice), murines (e.g., mice), canines (e.g., dogs), primates, anthropoids (e.g., monkeys or apes), monkeys (e.g., marmosets, baboons), apes (e.g., gorillas, chimpanzees, orangutans, gibbons). In some embodiments, “patient” is a human.

As used herein, “treatment” refers to therapeutic treatment or palliative measures. When specific conditions are involved, treatment refers to: (1) relieving one or more biological manifestations of a disease or a disorder, (2) interfering with (a) one or more points in a biological cascade that causes or contributes to a disorder or (b) one or more biological manifestations of a disorder, (3) ameliorating one or more symptoms, effects, or side effects associated with a disorder, or one or more symptoms, effects, or side effects associated with a disorder or treatment thereof, or (4) slowing the progression of one or more biological manifestations of a disease or a disorder. “Treatment” may also refer to an increase in survival compared to expected survival without receiving the treatment.

As used herein, “prevention” refers to a reduction in the risk of acquiring or developing a disease or a disorder.

In some embodiments, the pharmaceutically acceptable salt of furmonertinib is a mesilate salt of furmonertinib, i.e., furmonertinib mesilate.

In some embodiments, the pharmaceutical composition of the present disclosure is present in the formulation form of a tablet or a capsule.

In some embodiments, in each unit formulation (such as a tablet or a capsule) of the pharmaceutical composition, the content of furmonertinib or a pharmaceutically acceptable salt thereof is 10 mg-400 mg, such as 20 mg-320 mg. As the specific content, for example, it can be 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. In one embodiment, it can be 20 mg, 40 mg, 80 mg, 160 mg, 240 mg or 320 mg, such as 40 mg or 80 mg, such as 40 mg.

In some embodiments, in the pharmaceutical composition, the content of furmonertinib or a pharmaceutically acceptable salt thereof is 80 mg-400 mg, for example, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As exemplary embodiments, it can be 80 mg, 160 mg, 240 mg or 320 mg, such as 80 mg, 160 mg or 240 mg, such as 240 mg.

In some embodiments, when the pharmaceutical composition is used for treating and/or preventing a disease mediated by HER2 exon 20 insertion mutation and/or EGFR rare mutation, the composition is administered to a patient such that the dose of furmonertinib or a pharmaceutically acceptable salt thereof is 80 mg-400 mg. As the specific dose, for example, it can be 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As exemplary embodiments, it can be 80 mg, 160 mg, 240 mg or 320 mg, such as 80 mg, 160 mg or 240 mg, such as 240 mg. In an embodiment of the present disclosure, the dose is a daily dose.

In some embodiments, the content of furmonertinib or a pharmaceutically acceptable salt thereof in the pharmaceutical composition refers to the total amount of furmonertinib or a pharmaceutically acceptable salt thereof in the pharmaceutical composition taken by a patient when said pharmaceutical composition is administered to the patient. For example, when a pharmaceutical composition is present in the formulation form of a tablet or a capsule, the content of furmonertinib or a pharmaceutically acceptable salt thereof in said pharmaceutical composition refers to the total amount of furmonertinib or a pharmaceutically acceptable salt thereof in all formulations (such as tablets or capsules) when the formulations (such as tablets or capsules) are administered.

It will be appreciated by those skilled in the art that when a patient is administered, the daily dose of furmonertinib or a pharmaceutically acceptable salt thereof is not less than the content of furmonertinib or a pharmaceutically acceptable salt thereof in per unit formulation. Those skilled in the art can calculate the total amount of the formulations that is necessary to be administered per day based on the daily dose of furmonertinib or a pharmaceutically acceptable salt thereof and the content of furmonertinib or a pharmaceutically acceptable salt thereof in each unit formulation. For example, when furmonertinib or a pharmaceutically acceptable salt thereof is contained in tablets and the content of furmonertinib or a pharmaceutically acceptable salt thereof in each unit formulation (each tablet) is 40 mg, and when the daily dose of furmonertinib or a pharmaceutically acceptable salt thereof is 240 mg, the total amount of the formulations (tablets) that is necessary to be administered per day is 6 tablets.

In some embodiments, the pharmaceutical composition is administered 1, 2 or 3 times per day, such as once per day, for the treatment and/or prevention of a disease mediated by HER2 exon 20 insertion mutation and/or EGFR rare mutation.

In some embodiments, the pharmaceutical composition may further comprise at least one second therapeutic agent. As the second therapeutic agent, it may be selected from chemotherapeutic drug, targeted antitumor drug, antibody drug and immunotherapeutic drug.

In some embodiments, as said chemotherapeutic drug, the following can be exemplified: one or more of platinum drug (for example oxaliplatin, cisplatin, carboplatin, nedaplatin, dicycloplatin, lobaplatin, triplatinum tetranitrate, phenanthreneplatin, picoplatin, miriplatin, satraplatin), fluoropyrimidine derivative (for example gemcitabine, capecitabine, ancitabine, fluorouracil, tegadifur, doxifluridine, tegafur, carmofur, trifluridine, tegafur), camptothecins (for example camptothecin, hydroxycamptothecine, 9-amino camptothecin, 7-ethyl camptothecin, irinotecan, topotecan), taxels (for example paclitaxel, albumin-bound paclitaxel and docetaxel), vinblastines (vinorelbine, vinblastine, vincristine, vindesine, vinflunine), anthracenes (epirubicin, amycin, rubidomycin, pirarubicin, amrubicin, idarubicin, mitoxantrone, aclarubicin, valrubicin, zorubicin, pixantrone), antibiotics, podophyllums, antimetabolite antitumor drug, pemetrexed, carmustine, melphalan, etoposide, teniposide, mitomycin, iphosphamide, cyclophosphamide, azacitidine, methotrexate, bendamustine, liposome amycin, actinomycin D (dactinomycin), bleomycin, pingyangmycin, temozolomide, decarbazine, peplomycin, eribulin, plinabulin, sapacitabine, treosulfan, 153Sm-EDTMP, and encequidar.

In some embodiments, said second therapeutic agent is one or more of platinum drug, and said platinum drug includes, but is not limited to Cisplatin, Carboplatin, Nedaplatin, oxaliplatin, triplatinum tetranitrate, phenanthreneplatin, picoplatin, satraplatin, miriplatin, Lobaplatin and the like.

In some embodiments, said chemotherapeutic drug is selected from one or more of etoposide, irinotecan, cisplatin, carboplatin, lobaplatin, nedaplatin, topotecan, paclitaxel, docetaxel, temozolomide, vinorelbine, gemcitabine, cyclophosphamide, amycin, vincristine, bendamustine, pharmorubicin, methotrexate, amrubjcin, tegafur, gimeracil, oteracil, tegafur.

In some embodiments, as the targeted antitumor drug, protein kinase inhibitors can be enumerated. Among them, the protein kinase inhibitors include but are not limited to tyrosine kinase inhibitors, serine and/or threonine kinase inhibitors, and poly ADP-ribose polymerase (PARP) inhibitors. The targets of the inhibitors include but are not limited to Fascin-1 protein, HDAC (histone deacetylase), Proteasome, CD38, SLAMF7 (CS1/CD319/CRACC), RANKL, EGFR (epidermal growth factor receptor), anaplastic lymphoma (ALK), METgene, ROS1gene, HER2gene, RETgene, BRAFgene, PI3K signal pathway, DDR2 (discoidin domain receptor 2) gene, FGFR1 (fibroblast growth factor receptor 1), NTRK1 (neurotrophic tyrosine kinase type 1 receptor) gene, and KRASgene. The targets of the targeted antitumor drug also include COX-2 (epoxidase-2), APE1 (apurinic-apyrimidinic endonuclease), VEGFR (vascular endothelial growth factor receptor), CXCR-4 (chemokine receptor-4), MMP (matrix metalloproteinase), IGF-1R (insulin-like growth factor receptor), Ezrin, PEDF (pigmented epithelial derived factor), AS, ES, OPG (bone protective factor), Src, IFN, ALCAM (activated leukocyte cell adhesion molecule), HSP, JIP1, GSK-3β (Glycogen Synthetic Kinase 3β), CyclinD1 (cell cycle regulator protein), CDK4 (cyclin-dependent kinase), TIMP1 (tissue metalloproteinase inhibitor), THBS3, PTHR1 (parathyroid hormone-related protein receptor 1), TEM7 (human tumor vascular endothelial marker 7), COPS3, and cathepsin K. The targeted antitumor drug that can be enumerated includes but is not limited to one or more of Imatinib, Sunitinib, Nilotinib, bosutinib, Saracatinib, Pazopanib, Trabectedin, Regorafenib, Cediranib, Bortezomib, Panobinostat, Carfilzomib, Ixazomib, apatinib, Erlotinib, Afatinib, Crizotinib, Ceritinib, Vemurafenib, Dabrafenib, Cabozantinib, Gefitinib, Dacomitinib, Almonertinib, Osimertinib, Olmutinib, Alectinib, Brigatinib, Lorlatinib, Trametinib, Larotrectinib, icotinib, Lapatinib, Vandetanib, Selumetinib, Sorafenib, Olmutinib, Savolitinib, Fruquintinib, Entrectinib, Dasatinib, Ensartinib, Lenvatinib, itacitinib, Pyrotinib, Binimetinib, Erdafitinib, Axitinib, Neratinib, Cobimetinib, Acalabrutinib, Famitinib, Masitinib, Ibrutinib, Anlotinib, rociletinib, nintedanib, Revlimid, LOXO-292, Vorolanib, bemcentinib, capmatinib, entrectinib, TAK-931, ALT-803, palbociclib, famitinib L-malate, LTT-462, BLU-667, ningetinib, tipifarnib, poziotinib, DS-1205c, capivasertib, SH-1028, Metformin, seliciclib, OSE-2101, APL-101, berzosertib, idelalisib, lerociclib, ceralasertib, PLB-1003, tomivosertib, SKLB-1028, D-0316, LY-3023414, allitinib, MRTX-849, AP-32788, AZD-4205, lifirafenib, vactosertib, mivebresib, napabucasin, sitravatinib, TAS-114, molibresib, CC-223, rivoceranib, CK-101, LXH-254, simotinib, GSK-3368715, TAS-0728, masitinib, tepotinib, HS-10296, AZD-4547, merestinib, olaptesed pegol, galunisertib, ASN-003, gedatolisib, defactinib, lazertinib, CKI-27, S-49076, BPI-9016M, RF-A-089, RMC-4630, AZD-3759, antroquinonol, SAF-189s, AT-101, TTI-101, naputinib, LNP-3794, HH-SCC-244, ASK-120067, CT-707, epitinib succinate, tesevatinib, SPH-1188-11, BPI-15000, copanlisib, niraparib, olaparib, veliparib, talazoparib tosylate, DV-281, Siremadlin, Telaglenastat, MP-0250, GLG-801, ABTL-0812, bortezomib, tucidinostat, vorinostat, resminostat, epacadostat, tazemetostat, entinostat, mocetinostat, quisinostat, LCL-161, and KML-001. In some embodiments, the targeted antitumor drug is one or more of Sorafenib, Erlotinib, Afatinib, Crizotinib, Ceritinib, Vemurafenib, Dabrafenib, Cabozantinib, Gefitinib, Dacomtinib, Osimertinib, Alectinib, Brigatinib, Lorlatinib, Trametinib, Larotrectinib, Icotinib, Lapatinib, Vandetanib, Selumetinib, Olmutinib, Savolitinib, Fruquintinib, Entrectinib, Dasatinib, Ensartinib, Lenvatinib, Itacitinib, Pyrotinib, Binimetinib, Erdafitinib, Axitinib, Niratinib, Cobimetinib, Acalabrutinib, Famitinib, Masitinib, Ibrutinib, Anlotinib, Nintedanib.

In some embodiments, the second therapeutic agent is an antibody drug. Among others, the targets aimed by the antibody drug include but are not limited to any one or more of PD-1, PD-L1, cytotoxic T-lymphocyte antigen 4 (CTLA-4), platelet-derived growth factor receptor α (PDGFR-α), vascular endothelial growth factor (VEGF), human epidermal growth factor receptor-2 (HER2), epidermal growth factor receptor (EGFR), ganglioside GD2, B-cell surface protein CD20, B-cell surface protein CD52, B-cell surface protein CD38, B-cell surface protein CD319, B-cell surface protein CD30, and B-cell surface protein CD19/CD3.

In some embodiments, the antibody drug is an inhibitor for the interaction between the PD-1 receptor and its ligand PD-L1; in an embodiment of the present disclosure, the antibody drug is cytotoxic T-lymphocyte antigen 4 inhibitor. In an embodiment of the present disclosure, the antibody drug is platelet-derived growth factor receptor α (PDGFR-α) inhibitor.

In some embodiments, the inhibitor for the interaction between the PD-1 receptor and its ligand PD-L1 is an antibody or its antigen-binding portion that binds to the programmed death receptor 1 (PD-1) and/or inhibits the activity of PD-1, or an antibody or its antigen-binding portion that binds to the programmed death ligand 1 (PD-L1) and/or inhibits the activity of PD-L1, for example, an anti-PD-1 antibody or an anti-PD-L1 antibody. In an embodiment of the present disclosure, the antibody or its antigen-binding portion is (a) an anti-PD-1 monoclonal antibody or its antigen-binding fragment, which specifically binds to human PD-1 and blocks the binding between human PD-L1 and human PD-1; or (b) an anti-PD-L1 monoclonal antibody or its antigen-binding fragment, which specifically binds to human PD-L1 and blocks the binding between human PD-L1 and human PD-1.

In some embodiments, the anti-PD-1 or PD-L1 antibody is an anti-PD-1 or PD-L1 monoclonal antibody.

In some embodiments, the anti-PD-1 or PD-L1 antibody is a human antibody or a murine antibody.

In some embodiments, the anti-PD-1 antibody can be selected from any one or more of Nivolumab, Pembrolizumab, Durvalumab, Toripalimab (JS-001), Sintilimab (IBI308), Camrelizumab, Tislelizumab (BGB-A317), Geptanolimab (GB226), Lizumab (LZM009), HLX-10, BAT-1306, AK103 (HX008), AK104 (Akesobio), CS1003, SCT-I10A, F520, SG001, and GLS-010.

In some embodiments, the anti-PD-L1 antibody can be selected from any one or more of Atezolizumab, Avelumab, Durvalumab, KL-A167, SHR-1316, BGB-333, JS003, STI-A1014(ZKAB0011), KN035, MSB2311, HLX-20, and CS-1001.

In some embodiments, the anti-PD-1 antibody is Toripalimab.

In some embodiments, the anti-PD-1 antibody is Pembrolizumab.

In some embodiments, the cytotoxic T-lymphocyte antigen 4 (CTLA-4) inhibitor is an anti-CTLA-4 antibody, in an embodiment of the present disclosure, the anti-CTLA-4 antibody is an anti-CTLA-4 monoclonal antibody.

In some embodiments, the anti-CTLA-4 antibody can be selected from any one or more of Ipilimumab, Tremelimumab, AGEN-1884, BMS-986249, BMS-986218, AK-104, and IBI310.

In some embodiments, the anti-CTLA-4 antibody is Ipilimumab.

In some embodiments, the platelet-derived growth factor receptor α (PDGFR-α) inhibitor is an anti-PDGFR α antibody. In an embodiment of the present disclosure, the anti-PDGFRα antibody is an anti-PDGFRα monoclonal antibody.

In some embodiments, the anti-PDGFRα antibody is Olaratumab.

In some embodiments, the antibody drug can also include, but are not limited to any one or more of Bevacizumab, Ramucirumab, Pertuzumab, Trastuzmab, Cotuximab, Nimotuzumab, Panitumumab, Necitumumab, Dinutuximab, Rituximab, Ibritumomab, Ofatumumab, Obinutuzumab, Alemtuzumab, Daratumumab, Gemtuzumab, Elotuzumab, Brentuximab, Inotuzumab Ozogamicin, Blinatumomab.

In some embodiments, as immunotherapeutic drug, the following can be enumerated: one or more interferon (interferon α, interferon α-1b, interferon α-2b), interleukin, temsirolimus, everolimus, ridaforolimus, and temsirolimus.

In some embodiments, when a second therapeutic agent is used, the amount of the second therapeutic agent can be adjusted as desired by those skilled in the art.

In some embodiments, use of furmonertinib or a pharmaceutically acceptable salt thereof in manufacture of a medicament for treating and/or preventing a disease mediated by HER2 exon 20 insertion mutation and/or EGFR rare mutation is provided.

In some embodiments, use of furmonertinib or a pharmaceutically acceptable salt thereof in combination of at least one second therapeutic agent in manufacture of a medicament for treating and/or preventing a disease mediated by HER2 exon 20 insertion mutation and/or EGFR rare mutation is provided.

In some embodiments, use of the above-mentioned pharmaceutical composition in manufacture of a medicament for treating and/or preventing a disease mediated by the HER2 exon 20 insertion mutation and/or EGFR rare mutation is provided.

In some embodiments, in the above use of the present disclosure, the pharmaceutically acceptable salt of furmonertinib is a mesilate salt of furmonertinib, i.e., furmonertinib mesilate.

In some embodiments, in the use described herein, the pharmaceutical composition of the present disclosure is present in the formulation form of a tablet or a capsule.

In some embodiments, in the use described herein, in each unit formulation (such as a tablet or a capsule), the content of furmonertinib or a pharmaceutically acceptable salt thereof is 10 mg-400 mg, such as 20 mg-320 mg. As the specific content, it can be for example 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary specific content, it can be 20 mg, 40 mg, 80 mg, 160 mg, 240 mg or 320 mg, such as 40 mg or 80 mg, such as 40 mg.

In some embodiments, in the use described herein, in said pharmaceutical composition, the content of furmonertinib or a pharmaceutically acceptable salt thereof is 80 mg-400 mg, for example 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary content, it can be 80 mg, 160 mg, 240 mg or 320 mg, such as 80 mg, 160 mg or 240 mg, such as 240 mg.

In some embodiments, in the use described herein, the pharmaceutical composition is administered to a patient such that the dose of furmonertinib or a pharmaceutically acceptable salt thereof is 80 mg-400 mg. As the specific dose, it can be for example 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary dose, it can be 80 mg, 160 mg, 240 mg or 320 mg, such as 80 mg, 160 mg or 240 mg, such as 240 mg. In an embodiment of the present disclosure, the dose is a daily dose.

In some embodiments, in the use described herein, the content of furmonertinib or a pharmaceutically acceptable salt thereof in the pharmaceutical composition refers to the total amount of furmonertinib or a pharmaceutically acceptable salt thereof in the pharmaceutical composition taken by a patient when said pharmaceutical composition is administered to the patient. For example, when a pharmaceutical composition is present in the formulation form of a tablet or a capsule, the content of furmonertinib or a pharmaceutically acceptable salt thereof in said pharmaceutical composition refers to the total amount of furmonertinib or a pharmaceutically acceptable salt thereof in all formulations (such as tablets or capsules) when the formulations (such as tablets or capsules) are administered.

It will be appreciated by those skilled in the art that in the use described herein, when a patient is administered, the daily dose of furmonertinib or a pharmaceutically acceptable salt thereof is not less than the content of furmonertinib or a pharmaceutically acceptable salt thereof in per unit formulation. Those skilled in the art can calculate the total amount of the formulations that is necessary to be administered per day based on the daily dose of furmonertinib or a pharmaceutically acceptable salt thereof and the content of furmonertinib or a pharmaceutically acceptable salt thereof in each unit formulation. For example, when furmonertinib or a pharmaceutically acceptable salt thereof is contained in tablets and the content of furmonertinib or a pharmaceutically acceptable salt thereof in each unit formulation (each tablet) is 40 mg, and when the daily dose of furmonertinib or a pharmaceutically acceptable salt thereof is 240 mg, the total amount of the formulations (tablets) that is necessary to be administered per day is 6 tablets.

In some embodiments, the disease mediated by HER2 exon 20 insertion mutation and/or EGFR rare mutation is cancer, for example lung cancer, and further can be non-small cell lung cancer (NSCLC).

In some embodiments, the disease mediated by HER2 exon 20 insertion mutation and/or EGFR rare mutation is locally advanced non-small cell lung cancer or metastatic non-small cell lung cancer.

In some embodiments, the disease mediated by HER2 exon 20 insertion mutation and/or EGFR rare mutation is a treatment-naive non-small cell lung cancer or a previously-treated non-small cell lung cancer.

As used herein, the term “treatment-naive” refers to a condition where before receiving the treatment with furmonertinib or a pharmaceutically acceptable salt thereof of the present disclosure, the treatment with another therapeutic agent (including but not limited to chemotherapeutic drug, targeted antitumor drug, antibody drug or immunotherapeutic drug) has not been used, or a condition where no systematic anti-tumor therapy has been taken. As used herein, the term “previously-treated” refers to a condition where before receiving the treatment with furmonertinib or a pharmaceutically acceptable salt thereof of the present disclosure, the treatment with another therapeutic agent (including but not limited to chemotherapeutic drug, targeted antitumor drug, antibody drug or immunotherapeutic drug) has been used, or a condition where a systematic anti-tumor therapy has been taken, but afterwards the disease has progressed. In the case of “previously-treated”, the patient may have developed the resistance to other therapeutic agents, or may not develop the drug resistance.

In some embodiments, the HER2 exon 20 insertion mutation is at least one selected from a group consisting of ERBB2 A775_G776insYVMA mutation, ERBB2 V777_G778insGC mutation, and ERBB2 P780_Y781insGSP mutation.

In an embodiment of the present invention, the EGFR rare mutation is at least one selected from a group consisting of EGFR G719S mutation, EGFR S768I mutation, EGFR G724S mutation, EGFR L861Q mutation, and EGFR G719S/T263P mutation.

In some embodiments, in the use described herein, the pharmaceutical composition may further comprise at least one second therapeutic agent.

In the use described herein, the second therapeutic agent can be selected from chemotherapeutic drug, targeted antitumor drug, antibody drug and immunotherapeutic drug.

In some embodiments, in the use described herein, the second therapeutic agent is the above-mentioned second therapeutic agent of the present disclosure.

In some embodiments, a method of treating and/or preventing a disease mediated by HER2 exon 20 insertion mutation and/or EGFR rare mutation is provided, comprising administering to a patient a therapeutically effective amount of furmonertinib or a pharmaceutically acceptable salt thereof.

In some embodiments, a method of treating and/or preventing a disease comprising administering to a patient with positive HER2 exon 20 insertion mutation and/or EGFR rare mutation a therapeutically effective amount of furmonertinib or a pharmaceutically acceptable salt thereof is provided.

In some embodiments, a method of treating locally advanced or metastatic non-small cell lung cancer is provided, comprising administering to a patient in need thereof a therapeutically effective amount of furmonertinib or a pharmaceutically acceptable salt thereof.

In some embodiments, a method of treating locally advanced or metastatic non-small cell lung cancer is provided, comprising administering to a patient with confirmed positive HER2 exon 20 insertion mutation and/or EGFR rare mutation a therapeutically effective amount of furmonertinib or a pharmaceutically acceptable salt thereof.

In some embodiments, a method of treating locally advanced or metastatic non-small cell lung cancer is provided, comprising administering to a patient harboring HER2 exon 20 insertion mutation and/or EGFR rare mutation a therapeutically effective amount of furmonertinib or a pharmaceutically acceptable salt thereof.

In some embodiments, a method of treating locally advanced or metastatic non-small cell lung cancer is provided, comprising administering to a patient with confirmed positive HER2 exon 20 insertion mutation and/or EGFR rare mutation who has received no prior systematic anti-tumor therapy a therapeutically effective amount of furmonertinib or a pharmaceutically acceptable salt thereof.

In some embodiments, a method of treating locally advanced or metastatic non-small cell lung cancer is provided, comprising administering to a patient with confirmed positive HER2 exon 20 insertion mutation and/or EGFR rare mutation who has progressive disease after receiving prior systematic anti-tumor therapy a therapeutically effective amount of furmonertinib or a pharmaceutically acceptable salt thereof.

In some embodiments of the treatment method, the furmonertinib or a pharmaceutically acceptable salt thereof is administrated at a dose of 80 mg-400 mg. As the specific dose, it can be for example 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary dose, it can be 80 mg, 160 mg, 240 mg or 320 mg, such as 80 mg, 160 mg or 240 mg, such as 240 mg. In an embodiment of the present disclosure, the dose is a daily dose.

In some embodiments of the treatment method, the frequency at which furmonertinib or a pharmaceutically acceptable salt thereof is administered to a patient is 1 time per day (qd), 2 times per day (bid), or 3 times per day (tid), such as 1 time per day.

In some embodiments of the treatment method, furmonertinib or a pharmaceutically acceptable salt thereof is administered to a patient under fasted state, such as under fasted state in the morning.

In some embodiments of the treatment method, furmonertinib or a pharmaceutically acceptable salt thereof is orally administered to a patient.

In some embodiments, in the treatment method described herein, furmonertinib is administered in the form of a mesilate salt.

In some embodiments of the treatment method, furmonertinib or a pharmaceutically acceptable salt thereof is administered in the formulation form of a tablet or a capsule.

In some embodiments of the treatment method, furmonertinib or a pharmaceutically acceptable salt thereof is administered to a patient in the form of each unit formulation. By adjusting the amount of unit formulation, the daily dose of furmonertinib or a pharmaceutically acceptable salt thereof is in the above range.

In some embodiments of the treatment method, in each unit formulation (such as a tablet or a capsule), the content of said furmonertinib or a pharmaceutically acceptable salt thereof is 10 mg-400 mg, such as 20 mg-320 mg. As the specific content, it can be for example 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary specific content, it can be 20 mg, 40 mg, 80 mg, 160 mg, 240 mg or 320 mg, such as 40 mg or 80 mg, such as 40 mg.

It will be appreciated by those skilled in the art that when a patient is administered, the daily dose of furmonertinib or a pharmaceutically acceptable salt thereof is not less than the content of furmonertinib or a pharmaceutically acceptable salt thereof in per unit formulation. Those skilled in the art can calculate the total amount of the formulations that is necessary to be administered per day based on the daily dose of furmonertinib or a pharmaceutically acceptable salt thereof and the content of furmonertinib or a pharmaceutically acceptable salt thereof in each unit formulation. For example, when furmonertinib or a pharmaceutically acceptable salt thereof is contained in tablets and the content of furmonertinib or a pharmaceutically acceptable salt thereof in each unit formulation (each tablet) is 40 mg, and when the daily dose of furmonertinib or a pharmaceutically acceptable salt thereof is 240 mg, the total amount of the formulations (tablets) that is necessary to be administered per day is 6 tablets.

In some embodiments of the treatment method, at least one second therapeutic agent can be further administered to a patient. In some embodiments of the treatment method, as the second therapeutic agent, it can be selected from chemotherapeutic drug, targeted antitumor drug, antibody drug and immunotherapeutic drug.

In some embodiments of the treatment method, the second therapeutic agent is the above-mentioned the second therapeutic agent of the present disclosure.

In some embodiments of the treatment method, the disease is cancer, for example lung cancer, and further can be non-small cell lung cancer (NSCLC).

In some embodiments of the treatment method, furmonertinib or a pharmaceutically acceptable salt thereof is administered to a patient before or after surgical resection of tumor.

In some embodiments of the treatment method, the disease is locally advanced non-small cell lung cancer or metastatic non-small cell lung cancer.

In some embodiments of the treatment method, the disease is a treatment-naive non-small cell lung cancer or a previously-treated non-small cell lung cancer.

In the above treatment method of the present disclosure, the HER2 exon 20 insertion mutation is at least one selected from a group consisting of ERBB2 A775_G776insYVMA mutation, ERBB2 V777_G778insGC mutation, and ERBB2 P780_Y781insGSP mutation.

In an embodiment of the present invention, the EGFR rare mutation is at least one selected from a group consisting of EGFR G719S mutation, EGFR S768I mutation, EGFR G724S mutation, EGFR L861Q mutation, and EGFR G719S/T263P mutation.

In some embodiments of the treatment method, the patient is a human patient.

In some embodiments of the treatment method, the patient is between age 18 and 75.

In some embodiments of the treatment method, the patient has histologically or cytopathologically confirmed primary non-small cell lung cancer (NSCLC) with predominant non-squamous cell histology prior to the start of treatment with furmonertinib or a pharmaceutically acceptable salt thereof.

In some embodiments of the treatment method, the patient has radiological disease progression following the last anti-tumor therapy prior to the start of treatment with furmonertinib or a pharmaceutically acceptable salt thereof.

In some embodiments of the treatment method, the patient has documented positive HER2 exon 20 insertion mutation and/or EGFR rare mutation by laboratory test prior to the start of treatment with furmonertinib or a pharmaceutically acceptable salt thereof.

In some embodiments of the treatment method, the patient has locally advanced non-small cell lung cancer or metastatic non-small cell lung cancer (NSCLC) and is confirmed to have radiological or pathological disease progression during or after the last systematic anti-tumor therapy prior to the start of treatment with furmonertinib or a pharmaceutically acceptable salt thereof.

In some embodiments of the treatment method, the patient has locally advanced non-small cell lung cancer or metastatic non-small cell lung cancer (NSCLC) and has received no prior systematic anti-tumor therapy prior to the start of treatment with furmonertinib or a pharmaceutically acceptable salt thereof.

In some embodiments of the treatment method, the patient has at least one measurable lesion prior to the start of treatment with furmonertinib or a pharmaceutically acceptable salt thereof.

In some embodiments of the treatment method, the patient has adequate organ function as shown by laboratory test prior to the start of treatment with furmonertinib or a pharmaceutically acceptable salt thereof.

In some embodiments of the treatment method, the patient is subjected to an ECOG PS (Eastern Cooperative Oncology Group performance status) score test, such as an ECOG PS score of 0-1, prior to the start of treatment with furmonertinib or a pharmaceutically acceptable salt thereof.

In some embodiments, the treatment method has an acceptable safety profile.

In some embodiments, the treatment method can provide the therapeutic efficacy of partial response (PR).

In some embodiments, the treatment method can provide the therapeutic efficacy of stable disease (SD).

In some embodiments, the treatment method can provide tumor shrinkage in target lesions.

In some embodiments of the treatment method, tumor shrinkage in target lesions is provided, as evaluated by tumor radiological examination, such as computed tomography (CT) and/or magnetic resonance imaging (MRI).

### BRIEF DESCRIPTION OF THE DRAWINGS

FIG. 1: The curves of tumor volume change in Test Example 2.

FIG. 2: The curves of body weight change rate in Test Example 2.

FIG. 3: The curves of tumor volume change in Test Example 3.

FIG. 4: The curves of body weight change in Test Example 3.

### EXAMPLES

### I. Preparation Examples

Preparation of furmonertinib mesilate, 40 mg, standard tablet

Formula: furmonertinib mesilate 46.76 mg, microcrystalline cellulose 44.73 mg, lactose 68.2 mg, croscarmellose sodium 13 mg, polyethylene glycol 4000 17.8 mg, colloidal silica 10.9 mg, sodium stearyl fumarate 2.7 mg, sodium chloride 8.67 mg, and 40 mg furmonertinib contained therein.

Process: sieving adjuvants and the active pharmaceutical ingredient for pretreatment and mixing uniformly, adding an appropriate amount of Polyethylene Glycol 4000 for wet granulation, sieving out for wet granulation, drying the wet granules, sieving out for granulation, adding colloidal silica and sodium stearyl fumarate and mixing uniformly, and then tabletting to obtain tablets.

### II. Activity Examples

### Test Example 1: Proliferation Inhibition Activity on Stably Transfected Cells Ba/F3 EGFR G719S, Ba/F3 EGFR G724S, Ba/F3 EGFR S768I, Ba/F3 EGFR L861Q, Ba/F3 EGFR G719S/T263P, Ba/F3 ERBB2 A775_G776insYVMA, Ba/F3 ERBB2 V777_G778insGC, and Ba/F3 ERBB2 P780_Y781insGSP

The proliferation inhibition activity of the compound (furmonertinib mesilate) on four cells of Ba/F3 EGFR G719S, Ba/F3 EGFR G724S, Ba/F3 EGFR S768I, and Ba/F3 EGFR L861Q stably expressing EGFR rare mutation proteins, one cell of Ba/F3 EGFR G719S/T263P stably expressing EGFR double mutation proteins, and three cells of Ba/F3 ERBB2 A775_G776insYVMA, Ba/F3 ERBB2 V777_G778insGC, and Ba/F3 ERBB2 P780_Y781insGSP stably expressing different ERBB2 exon 20 insertion mutation proteins in mouse pro-B cells Ba/F3 in vitro was determined by the CellTiter Glo method.

Cell source: Ba/F3 EGFR G719S, Ba/F3 EGFR G724S, Ba/F3 EGFR S768I, Ba/F3 EGFR L861Q, Ba/F3 EGFR G719S/T263P, Ba/F3 ERBB2 A775_G776insYVMA, Ba/F3 ERBB2 V777_G778insGC, Ba/F3 ERBB2 P780_Y781insGSP cells were provided by KYinno Biotechnology (Beijing) Co., Ltd.

Ba/F3 EGFR G719S, Ba/F3 EGFR G724S, Ba/F3 EGFR S768I, Ba/F3 EGFR L861Q, Ba/F3 EGFR G719S/T263P, Ba/F3 ERBB2 A775_G776insYVMA, Ba/F3 ERBB2 V777_G778insGC, Ba/F3 ERBB2 P780_Y781insGSP cells were cultivated in RPMI1640 complete culture medium containing 10% fetal bovine serum. Ba/F3 EGFR G719S, Ba/F3 EGFR G724S, Ba/F3 EGFR S768I, Ba/F3 EGFR L861Q, Ba/F3 EGFR G719S/T263P, Ba/F3 ERBB2 A775_G776insYVMA, Ba/F3 ERBB2 V777_G778insGC, Ba/F3 ERBB2 P780_Y781insGSP cells in the logarithmic growth phase were taken and inoculated in 96-well plates according to the cell density of 3000 cells/90 μL of complete culture medium/well, and the plates were placed in a constant temperature incubator containing 5% CO₂ at 37° C. and cultivated for 24 hours. The compound was dissolved in dimethyl sulfoxide (DMSO) in advance to prepare a 30 mM stock solution, and then the compound was successively diluted with DMSO and the complete culture medium. The 96-well plates inoculated with the cells were taken out, and 10 μL of different concentrations of the compound were added to each well to achieve final concentrations of 3000, 950, 300, 95.0, 30, 9.5, 3, 0.95, and 0.3 nM, three duplicate wells were set for each compound concentration, and a negative control (a cell-containing culture medium control) and a blank control (a cell-free culture medium control) were set, and the DMSO concentration in each well was 0.1%. The plates were placed in a constant temperature incubator containing 5% CO₂ at 37° C. and cultivated for 72 hours.

The CellTiter-Glo reagent (a luciferase ATP bioluminescence detection reagent, commercially available from Promega) was thawed, the 96-well plates inoculated with the cells were taken out from the CO₂ constant temperature incubator, and equilibrated to room temperature (about 30 minutes), 100 μL CellTiter-Glo reagent was added to each well, the cells were lyzed by shaking on an orbital shaker for 5 minutes, inoculated at room temperature for 20 minutes to wait for the luminescence intensity to stabilize, then the luminescence intensity (Lum) was determined with a microplate reader. The cell survival rate at each concentration of the compound was calculated.

$$Cell survival rate ( \% ) = ( {Lum}_{72 - hour compound administration group} - {Lum}_{blank control} ) / ( {Lum}_{72 - hour negative control group} - {Lum}_{blank control} ) \times 100 \% .$$

The data were analyzed using GraphPad Prism 7.0 software, fitted with nonlinear S-curve regression to give a dose-effect curve, and IC₅₀ values were calculated therefrom, as shown in Table 1.

TABLE 1

IC₅₀ (nM)

| | furmonertinib | |
|---|---|---|
| Stably transfected cells | mesilate | AZD9291 |
| Ba/F3 EGFR G719S | 19 | 68 |
| Ba/F3 EGFR G724S | 35 | 139 |
| Ba/F3 EGFR S768I | 33 | 137 |
| Ba/F3 EGFR L861Q | 13 | 41 |
| Ba/F3 EGFR G719S/T263P | 37 | 191 |
| Ba/F3 ERBB2 | 118 | 489 |
| A775_G776insYVMA | | |
| Ba/F3 ERBB2 V777_G778insGC | 25 | 77 |
| Ba/F3 ERBB2 P780_Y781insGSP | 33 | 96 |

The result showed that furmonertinib mesilate had good proliferation inhibition activity on Ba/F3 EGFR G719S, Ba/F3 EGFR G724S, Ba/F3 EGFR S768I, Ba/F3 EGFR L861Q, Ba/F3 EGFR G719S/T263P, Ba/F3 ERBB2 A775_G776insYVMA, Ba/F3 ERBB2 V777_G778insGC, Ba/F3 ERBB2 P780_Y781insGSP stably transfected cells.

### Test Example 2: Testing the Anti-Tumor Effect of Furmonertinib Mesilate in the Ba/F3 ERBB2 A775_G776insYVMA CDX Tumor Model

This study was used for evaluating and testing the antitumor effect of furmonertinib mesilate in Ba/F3 ERBB2 A775_G776insYVMA (a mouse pro-B cell Ba/F3 stably expressing ERBB2 exon 20 insertion mutation protein) subcutaneous xenografted BALB/c female nude mouse animal models.

Experimental animals: BALB/c nude mice, female, 8-9 weeks (mouse week-old when tumor cells were inoculated), and weighing 13.7-17.7 g, purchased from the Shanghai branch of Beijing Vital River Laboratory Animal Technology Co., Ltd.

Animal modeling and random grouping: Ba/F3 ERBB2 A775_G776insYVMA cells were cultivated, amplified to two T175 cm² culture flasks, cells were collected, and resuspended and counted in serum-free medium DMEM with the addition of a matrix gel at 1:1, and inoculated at 2×10⁶ cells/0.1 mL in Balb/c nude mouse right front scapular subcutaneous. When the average tumor volume was about 160 mm³, the animals were randomly grouped into three experiment groups according to the tumor size. Each group contained 6 mice. The grouping day was defined as Day 0, i.e., DO.

The experimental scheme: BALB/c nude mice were subcutaneously inoculated with Ba/F3 ERBB2 A775_G776insYVMA cells, and a cell line heterograft tumor model was established. The experiment was divided into 30 mg/kg group of AZD9291, 30 mg/kg group of furmonertinib mesilate and a vehicle control group, wherein each group contained 6 animals, orally administered with the administration volume of 10 uL/g, and the vehicle control group was administered with the same amount of vehicle, the administration was carried out once per day and lasted for two weeks. During the whole experiment, the body weight and the tumor sizes of the mice were measured twice each week, and whether or not the presence of toxic reactions was observed.

Tumor Volume (TV) was calculated by: TV=½×a×b×b, where a and b represented the length and the width of tumor, respectively.

$$Body weight change rate = ( Body weight / D 0 Body weight - 1 ) \times 100 \% .$$

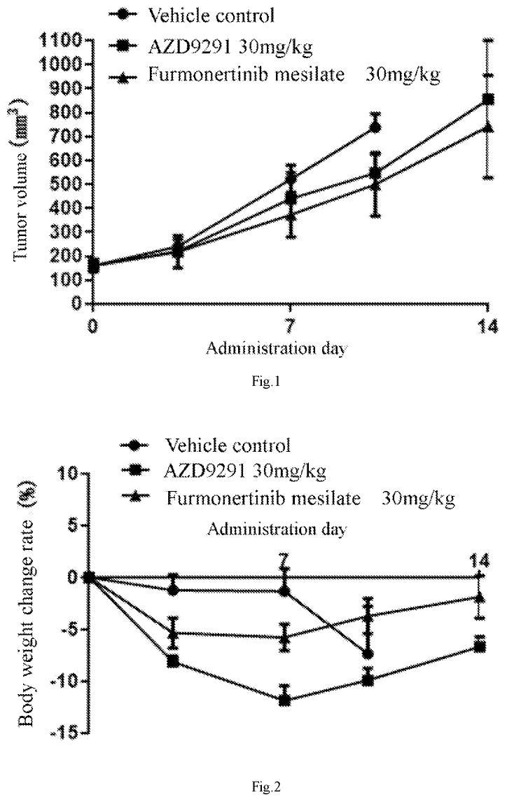

The curves for the tumor volume changes of three experimental groups were shown in FIG. 1, and the curves for the body weight change rate were shown in FIG. 2.

The result showed that furmonertinib mesilate showed good anti-tumor effect in Ba/F3 ERBB2 A775_G776insYVMA CDX subcutaneous xenografted BALB/c female nude mouse animal models, had less effect on the body weight of nude mice, and showed better safety.

### Test Example 3: Testing the Anti-Tumor Effect of Furmonertinib Mesilate in Ba/F3 ERBB2 V777_G778insGC(KC-1410) Cell Xenograft Model of Female Immune Deficiency Mice

This study was used for evaluate the anti-tumor efficacy of three compounds alone in the female (B-NDG) immune deficiency mice bearing tumors of Ba/F3 ERBB2 V777_G778insGC engineered cells.

Experimental animals: B-NDG mice, female, 6-8 weeks, and weighing 18-20 g.

Animal modeling and random grouping: Ba/F3 ERBB2 V777_G778insGC cells were cultivated, cells were collected, and re-suspended and counted in serum-free medium, the re-suspended cells with the addition of a matrigel at 1:1 were subcutaneous inoculated at 1×10⁶ cells/0.1 mL in B-NDG mice. When the average tumor volume was about 80-120 mm³, the animals were randomly grouped into four experiment groups according to the tumor size. Each group contained 12 mice. The grouping day was defined as Day 0, i.e., DO.

The experimental scheme: B-NDG mice were subcutaneously inoculated with Ba/F3 ERBB2 V777_G778insGC cells to establish a cell-line-derived xenograft model. The experiment was divided into 15 mg/kg group of furmonertinib mesilate, 30 mg/kg group of furmonertinib mesilate, 50 mg/kg group of furmonertinib mesilate and vehicle group, wherein each group contained 12 mice, orally administered with the administration volume of 10 uL/g, and the vehicle group was administered with the same amount of vehicle, the administration was carried out once per day and lasted for two weeks. During the whole experiment, the body weight and the tumor sizes of the mice were measured twice each week, and whether or not the presence of toxic reactions was observed.

Tumor Volume (TV) was calculated by: TV=½×a×b², where a was the long diameter of the tumor and b was the short diameter of the tumor.

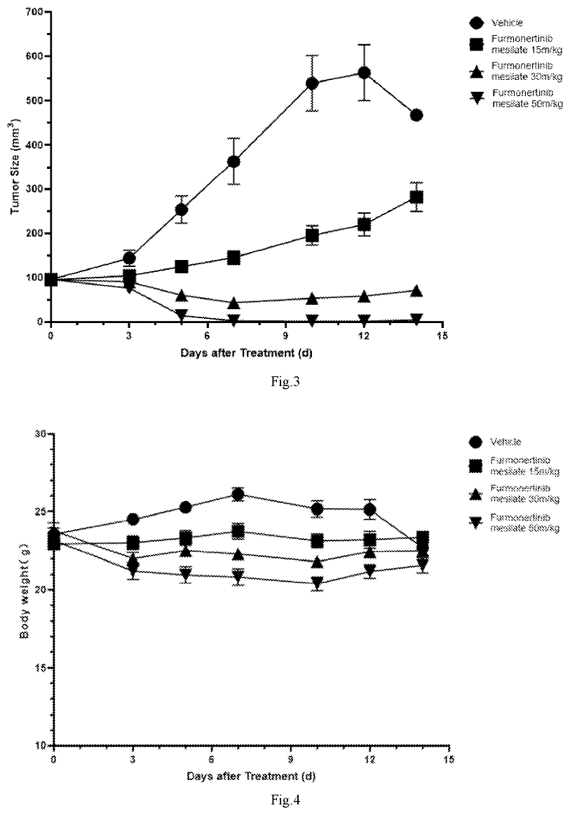

The curves for the tumor volume change of four experimental groups were shown in FIG. 3, and the curves for the body weight change of four experimental groups were shown in FIG. 4.

In summary, furmonertinib mesilate at 15 mg/kg produced moderate anti-tumor activity; furmonertinib mesilate at 30 mg/kg and furmonertinib mesilate at 50 mg/kg produced extremely significant anti-tumor activity, and the three furmonertinib mesilate groups had little effect on the body weight of mice, and showed good safety.

### Test Example 4: Studies of Furmonertinib in Patients with Advanced or Metastatic Non-Small Cell Lung Cancer (NSCLC) with Activating HER2 Mutations, Including Exon 20 Insertion Mutations

A clinical trial is performed to evaluate the safety, pharmacokinetics (PK), and antitumor activity of furmonertinib in at least 100 patients with advanced or metastatic NSCLC with activating HER2 mutations, including Exon 20 insertion mutations.

Patients are enrolled into 2 stages: Stage 1 (Dose Escalation and Backfill Cohorts) and Stage 2 (Dose Expansion). In each stage, previously treated NSCLC patients are administered with furmonertinib tablets.

The primary outcome measures include the incidence and severity of adverse events (AEs), as a measure of safety and tolerability of furmonertinib (the time frame is up to 36 months after first dose). The secondary outcome measures include, e.g., overall response rate (ORR), duration of response (DOR), progression free survival (PFS), overall survival, central nervous system (CNS) ORR, and central nervous system (CNS) DOR, up to 36 months after first dose.

Eligible patients include all sexes and are at least 18-year old. Inclusion criteria include, e.g.,
histologically or cytologically documented, locally advanced or metastatic NSCLC not amenable to curative surgery or radiotherapy;
disease that has progressed after at least one available standard therapy, or for whom standard therapy has proven to be ineffective or intolerable, or for whom a clinical trial of an investigational agent is a recognized standard of care;
patients with a history of treated CNS metastases or new asymptomatic CNS metastases detected at screening;
documented radiologic disease progression during or after the last systemic anti-cancer therapy before the first dose of furmonertinib;
for patients with EGFR mutations sensitive to osimertinib, the patient must have received osimertinib prior to study enrollment in regions where osimertinib is approved, including the US;
documented validated results from either local testing of blood or tumor tissue confirming the presence of HER2 Exon 20 insertion mutations;
and
the patient must have experienced disease progression or have intolerance to treatment with platinum-based chemotherapy.

Exclusion criteria include, e.g.,
treatment with chemotherapy, immunotherapy, biologic therapy or an investigational agent as anti-cancer therapy within 3 weeks or five half-lives prior to initiation of furmonertinib, whichever is shorter, or endocrine therapy within 2 weeks prior to initiation of furmonertinib;
radiation therapy as cancer therapy within 4 weeks prior to initiation of furmonertinib;
palliative radiation to bony metastases within 2 weeks prior to initiation of furmonertinib; and
adverse events from prior anti-cancer therapy that have not resolved to Grade ≤1 except for alopecia or Grade ≤2 peripheral neuropathy.

### INDUSTRIAL APPLICABILITY

The present disclosure provides a pharmaceutical composition containing a therapeutically effective amount of furmonertinib or a pharmaceutically acceptable salt thereof and optionally a pharmaceutically acceptable carrier, use of furmonertinib or a pharmaceutically acceptable salt thereof, and said pharmaceutical composition in manufacture of a medicament for treating and/or preventing a disease mediated by HER2 exon 20 insertion mutation and/or EGFR rare mutation. The present disclosure also provides a method of treating and/or preventing a disease mediated by HER2 exon 20 insertion mutation and/or EGFR rare mutation, wherein a therapeutically effective amount of furmonertinib or a pharmaceutically acceptable salt thereof is administered to a patient. The pharmaceutical composition of the present disclosure shows an excellent therapeutic effect on disease mediated by HER2 exon 20 insertion mutation and/or EGFR rare mutation (for example, non-small cell lung cancer (NSCLC)) with little side effects and excellent safety.

1-82. (canceled)

83. A method of treating advanced or metastatic non-small cell lung cancer (NSCLC) in a patient with an Epidermal Growth Factor Receptor (EGFR) rare mutation, comprising administering to the patient furmonertinib or a pharmaceutically acceptable salt thereof in an amount effective to inhibit proliferation of cancer cells with the EGFR rare mutation.

84. The method of claim 83, wherein the EGFR rare mutation is G719S, G724S, S768I, or L861Q.

85. The method of claim 83, wherein the furmonertinib or the pharmaceutically acceptable salt thereof is administered at a daily dose of about 80 mg.

86. The method of claim 83, wherein the furmonertinib or the pharmaceutically acceptable salt thereof is administered at a daily dose of about 160 mg.

87. The method of claim 83, wherein the furmonertinib or the pharmaceutically acceptable salt thereof is administered at a daily dose of about 240 mg.

88. The method of claim 83, wherein the furmonertinib or the pharmaceutically acceptable salt thereof is administered orally.

89. The method of claim 83, wherein the furmonertinib or the pharmaceutically acceptable salt thereof is administered orally, once daily.

90. the method of claim 83, wherein the patient has received no prior systematic anti-tumor therapy.

91. The method of claim 83, wherein the patient has progressive disease after receiving prior systematic anti-tumor therapy.

92. The method of claim 83, wherein the patient has histologically or cytopathologically confirmed primary NSCLC with predominant non-squamous cell histology.

93. The method of claim 83, wherein the NSCLC is advanced or metastatic NSCLC.

94. A method of inhibiting proliferation of cancer cells, comprising contacting the cancer cells with an effective amount of furmonertinib or a pharmaceutically acceptable salt thereof, wherein the cancer cells comprise an Epidermal Growth Factor Receptor (EGFR) rare mutation.

95. The method of claim 94, wherein the EGFR rare mutation is G719S, G724S, S768I, or L861Q.

96. The method of claim 94, wherein the method inhibits proliferation more effectively than contacting the cancer cells with AZD9291 at the same concentration as the furmonertinib.

97. The method of claim 94, wherein the furmonertinib inhibits proliferation at an IC₅₀ between about 10 nM and about 40 nM.

98. A method of treating advanced or metastatic non-small cell lung cancer (NSCLC) in a patient with a Human Epidermal growth factor Receptor-2 (HER2) exon 20 insertion, comprising administering to the patient furmonertinib or a pharmaceutically acceptable salt thereof in an amount effective to inhibit proliferation of cancer cells with a HER2 exon 20 insertion.
